(19) United States
(12) Patent Application Publication
(10) Pub. No.: US 2021/0259545 A1
(43) Pub. Date: Aug. 26, 2021

(54) OPHTHALMIC PHOTOGRAPHY APPARATUS AND OPHTHALMIC PHOTOGRAPHY SYSTEM

(71) Applicant: nanolux co. ltd., Ibaraki (JP)

(72) Inventors: Hironari TAKEHARA, Ikoma-shi, Nara (JP); Jun OHTA, Ikoma-shi, Nara (JP); Hirofumi SUMI, Tsukuba-shi, Ibaraki (JP); Motoshi SOBUE, Tsukuba-shi, Ibaraki (JP)

(21) Appl. No.: 17/251,206

(22) PCT Filed: Jun. 14, 2019
(86) PCT No.: PCT/JP2019/024616

§ 371 Date: Dec. 11, 2020

(30) Foreign Application Priority Data
Jun. 14, 2018 (JP) 2018-113280

(51) Int. Cl. A61B 3/12; A61B 3/14; A61B 3/00

(52) U.S. Cl. CPC A61B 3/12; A61B 3/0025; A61B 3/14

(57) ABSTRACT

An ophthalmic photography apparatus is provided with an irradiation optical system that irradiates a subject's eye with near-infrared light containing two or more wavelength components, a light receiving optical system that forms an image by concentrating reflected light derived from the near-infrared light reflected off a fundus or interior of the subject's eye, an imaging unit that outputs an image signal for each wavelength component by taking a fundus image or an intraocular image formed by the light receiving optical system, and an image generation unit that generates a fundus image or an intraocular image of the subject's eye by combining image signals outputted from the imaging unit.

### RELATED APPLICATIONS

The present application is a National Phase of International Application Number PCT/JP2019/024616, filed Jun. 14, 2019, which claims priority to Japanese Application Number 2018-113280, filed Jun. 14, 2018.

### TECHNICAL FIELD

The present invention relates to an ophthalmic photography apparatus that photographs a fundus of a subject and an ophthalmic photography system including such an apparatus.

### BACKGROUND ART

In general, a fundus examination is done by irradiating a subject's eye with visible light, detecting reflected light from the fundus, and making an image. Meanwhile, since photographing based on irradiation with visible light is glaring and places a burden on the subject, there have been proposed fundus photography methods that involve the concomitant use of visible light and non-visible light, such as infrared light, that is not sensed by a human eye (See, for example, PTLs 1 to 3.).

PTL 1 discloses a fundus camera including an illuminating light source including visible light and infrared light and an imaging element having sensitivity to a visible range and an infrared range. This fundus camera is configured to perform an infrared test emission to set an amount of emission of visible light illumination for use in photographing. Further, PTL 2 discloses a fundus photography apparatus configured to observe the front part of an eye with infrared light having a center wavelength of 940 nm and make a fundus observation through visible light. Furthermore, PTL 3 discloses a fundus photography system configured to sharpen a fundus image by combining an image taken by irradiation with infrared light and an image taken by irradiation with visible light.

Conventionally, there have also been proposed methods for photographing a fundus with infrared light alone (See, for example, PTLs 4 and 5). PTL 4 discloses a fundus photography apparatus configured to photograph a fundus of an identical subject in different states of polarization by irradiating the subject's eye with circularly polarized infrared light, converting a reflection of the circularly polarized infrared light into linearly polarized light, taking a photograph for each direction of polarization. Further, PTL 5 discloses a fundus photography apparatus configured to radiate light in an infrared region of 700 to 1000 nm and obtain a fundus spectroscopic image from spectroscopic data on a reflection of the light.

Further, there have recently been proposed an ophthalmic photography apparatus (see, for example, PTL 6) configured to take a moving image of a fundus as well as a still image and a system (see, for example, PTL 7) configured to measure and display fundus blood flow information or other information in addition to a photographed fundus morphological image.

### CITATION LIST

### Patent Literatures

PTL 1: Japanese Unexamined Patent Application Publication No. 2005-279154

PTL 2: Japanese Unexamined Patent Application Publication No. 2017-100013

PTL 3: Japanese Unexamined Patent Application Publication No. 2013-198587

PTL 4: Japanese Unexamined Patent Application Publication No. 2012-34724

PTL 5: Japanese Unexamined Patent Application Publication No. 2005-296400

PTL 6: Japanese Unexamined Patent Application Publication No. 2018-089480

PTL 7: Japanese Unexamined Patent Application Publication No. 2019-042263

### SUMMARY OF INVENTION

### Technical Problem

However, the fundus photography apparatuses disclosed in PTLs 1 to 3 listed above fail to reduce glare during photographing, as they also perform visible light photographing, and make it difficult to consecutively take a plurality of photographs, as doing so places a burden on a subject. Meanwhile, the apparatuses disclosed in PTLs 4 and 5 can reduce glare during photographing, as they take photographs with infrared light alone. However, the apparatus disclosed in PTL 4 is undesirably unable to obtain wavelength-dependent information, and the apparatus disclosed in PTL 5 undesirably takes a long time to take a photograph, as it acquires an image by scanning.

To address these problems, the present invention has as an object to provide an ophthalmic photography apparatus and an ophthalmic photography system that make it possible to, without placing a burden on a subject, obtain a fundus image that is similar to a conventional color photography based on visible light and other ophthalmic information.

### Solution to Problem

According to the present invention, there is provided an ophthalmic photography apparatus including an irradiation optical system that irradiates a subject's eye with near-infrared light containing two or more wavelength components, a light receiving optical system that forms an image by concentrating reflected light derived from the near-infrared light reflected off a given location in a fundus or interior of the subject's eye, an imaging unit that outputs an image signal for each wavelength component by taking a fundus image or an intraocular image formed by the light receiving optical system, and an image generation unit that generates a fundus image or an intraocular image of the subject's eye by combining image signals outputted from the imaging unit.

The imaging unit may include an imaging element that has two or more types of pixels having different detection wavelengths and that simultaneously detects two or more near-infrared lights having different center wavelengths.

Note here that the imaging element may have a first near-infrared pixel that receives a first near-infrared light, a second near-infrared pixel that receives a second near-infrared light differing in center wavelength from the first near-infrared light, and a third near-infrared pixel that receives a third near-infrared light differing in center wavelength from the first near-infrared light and the second near-infrared light.

The aforementioned pixels may be provided on an identical element.

Alternatively, the aforementioned pixels may be provided on different elements for each detection wavelength, and a spectral element may be provided that splits light reflected off the fundus or interior of the subject's eye and emits the light thus split separately toward the pixels.

Further, the imaging element may further have a first visible pixel that receives a first visible light, a second visible pixel that receives a second visible light differing in center wavelength from the first visible light, and a third visible pixel that receives a third visible light differing in center wavelength from the first visible light and the second visible light. In that case, the irradiation optical system may radiate visible light either together with near-infrared light or separately from near-infrared light, and the light receiving optical system may form an image of reflected light derived from the visible light.

The irradiation optical system may include a light source that simultaneously emits two or more near-infrared lights having different center wavelengths. It should be noted that the term “simultaneously” here does not need to be simultaneous in a strict sense but also encompasses a case where there is a time lag that can be allowed for a fundus image or an intraocular image. The same applies to the following description.

In that case, the irradiation optical system may be provided with a light pipe that homogenizes and emits incoming light, and the subject's eye may be irradiated with light from the light source via the light pipe.

The ophthalmic photography apparatus of the present invention may further include a data storage unit in which a fundus image and/or an intraocular image is/are stored and an image data processing unit that compares an image generated by the image generation unit with an image stored in the data storage unit.

According to the present invention, there is provided an ophthalmic photography system including the aforementioned ophthalmic photography apparatus and a server in which a fundus image and/or an intraocular image is/are stored, and compares an image taken by the ophthalmic photography apparatus with an image stored in the server.

### Advantageous Effects of Invention

The present invention makes it possible to obtain various types of ophthalmic information by taking a color fundus image and an intraocular image of a subject's eye with a lighter burden than visible light photographing.

### BRIEF DESCRIPTION OF DRAWINGS

The patent or application file contains at least one drawing and/or photograph executed in color. Copies of this patent or patent application publication with color drawing(s) and/or photograph(s) will be provided by the Office upon request and payment of the necessary fee.

FIG. 1 is a diagram schematically showing a configuration of an ophthalmic photography apparatus of a first embodiment of the present invention.

FIG. 2 is a diagram showing an example configuration of a light source 21 shown in FIG. 1.

FIG. 3 is a diagram schematically showing an example configuration of an imaging unit 4 shown in FIG. 1.

FIG. 4 is a diagram schematically showing another example configuration of the imaging unit 4 shown in FIG. 1.

FIG. 5 is a diagram schematically showing another example configuration of the imaging unit 4 shown in FIG. 1.

FIG. 6 is a diagram showing an example pixel arrangement of an imaging element having two or more types of pixels having different detection wavelengths.

FIG. 7 is a diagram showing the detection wavelength of each pixel shown in FIG. 6.

FIG. 8 is a diagram showing an example pixel arrangement of an imaging element that is capable of detecting both visible light and near-infrared light.

FIG. 9 is a diagram showing the detection wavelength of the imaging element shown in FIG. 8.

FIGS. 10A to 10D show fundus images taken by the ophthalmic photography apparatus of the first embodiment of the present invention, FIG. 10A being a color composite image, FIGS. 10B to 10D being images based on near-infrared lights NIR1, NIR2 and NIR3 shown in FIG. 7, respectively.

FIG. 11 is a vascular observation image taken by the ophthalmic photography apparatus of the first embodiment of the present invention.

FIG. 12A and FIG. 12B illustrate perspective views schematically showing a configuration of an ophthalmic photography apparatus of a modification of the first embodiment of the present invention.

FIG. 13 is a diagram showing an overview of an ophthalmic photography system of a second embodiment of the present invention.

FIG. 14 is a flow chart showing how the ophthalmic photography system shown in FIG. 13 operates.

### DESCRIPTION OF EMBODIMENTS

In the following, embodiments for carrying out the present invention are described in detail with reference to the accompanying drawings. It should be noted that the present invention is not limited to the embodiments described below.

### First Embodiment

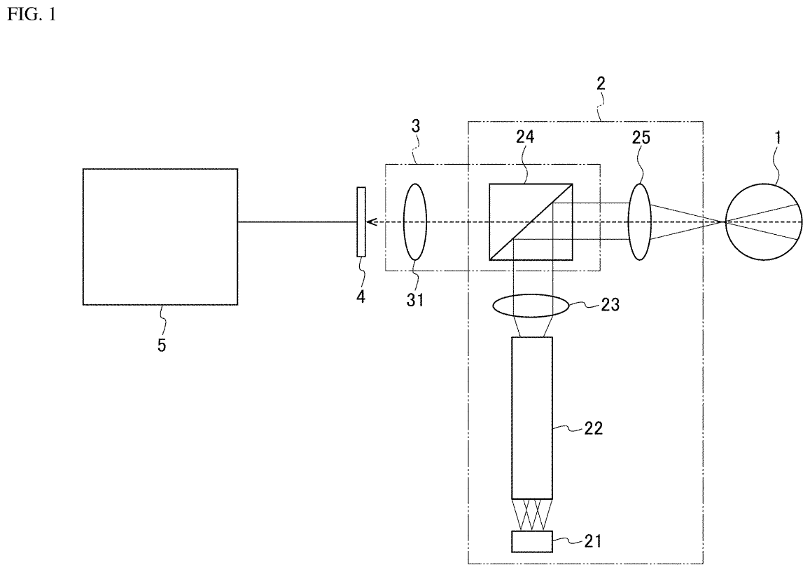

First, an ophthalmic photography apparatus according to a first embodiment of the present invention is described. FIG. 1 is a diagram schematically showing a configuration of the ophthalmic photography apparatus of the present embodiment of the present invention. As shown in FIG. 1, the ophthalmic photography apparatus of the present embodiment is provided with an irradiation optical system 2 that irradiates a subject's eye 1 with illuminating light, a light receiving optical system 3 that receives reflected light from the subject's eye 1, an imaging unit 4 that takes an fundus image or an intraocular image, an image generation unit 5 that generates a fundus image or an intraocular image from an image signal outputted from the imaging unit 4, or other constituent elements.

### [Irradiation Optical System 2]

The irradiation optical system 2 serves to irradiate the subject's eye 1 with near-infrared light containing two or more wavelength components, and is constituted by a light source 21, a light pipe 22, a condenser lens 23, a spectral element 24, an objective les 25, or other constituent elements. The light source 21 needs only emit near-infrared light containing two or more wavelength components, and usable examples of the light source 21 include a light source that is capable emitting a wide band of near-infrared lights ranging from 700 to 1100 nm, a light source obtained by combining a plurality of light-emitting diodes (LEDs) having different emission wavelengths, and similar light sources.

The light pipe 22 is an optical element that homogenizes incoming light by reflecting it more than once with the sides of a polygonal column or a polygonal cone and emits it, and is also called “homogenizer”. When obtained by combining a plurality of LEDs having different emission wavelengths, the light source 21 may develop unevenness in irradiation, depending on the arrangement and characteristics of the LEDs, misregistration of the light source 21, and the like. In such a case, placing the light pipe 22 between the light source 21 and the subject's eye 1 allows emission of light homogenized within the light pipe 22, thus making it possible to uniformly irradiate the subject's eye 1 with near-infrared light containing two or more wavelength components.

It should be noted that in a case where a light source that is capable of uniformly radiating near-infrared light containing two or more wavelength components is used, it is not necessary to provide the light pipe 22. Note here that examples of the light source that is capable of uniform irradiation include a light source in which a plurality of LEDs having different emission wavelengths are proximately arranged and sealed so that the locations of emission of two or more near-infrared lights having different center wavelengths are proximate to each other, a light source configured to emit a wide band of near-infrared lights using a short-wavelength LED and a near-infrared phosphor, and similar light sources. Further, placing a diffusing plate and/or a diaphragm at the back (emission side) of the light pipe 22 generates a simulated point light source, thus making it possible to further reduce unevenness in irradiation.

A point light source including LEDs has a risk of developing wavelength unevenness in illuminating light when the locations of emission of wavelengths of light are separate. Therefore, in the ophthalmic photography apparatus of the present embodiment, it is preferable to, in a case where LEDs are used as light sources, integrate and mount light sources of necessary wavelengths within a circle approximately several millimeters in diameter. This makes it possible to dispersedly illuminate the subject's eye 1 with a plurality of wavelengths of near-infrared light.

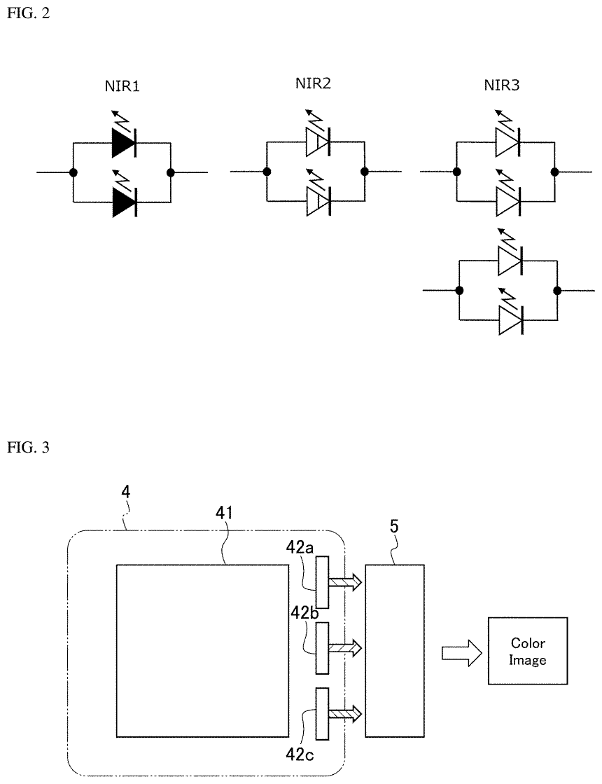

FIG. 2 is a diagram showing an example configuration of the light source 21 shown in FIG. 1. Detection of each near-infrared light by the imaging unit 4 may vary in sensitivity between wavelengths, depending on the type of the imaging element used. For example, in a case where the imaging element is formed on a Si substrate, sensitivity to light having a wavelength of 940 nm is lower by several tens of percent than to light having a wavelength of 800 nm. Therefore, in the ophthalmic photography apparatus of the present embodiment, as shown in FIG. 2 and Table 1 listed below, it is preferable that more LEDs that are compatible with the detection sensitivity of the imaging element and emit low-sensitivity wavelengths of light be mounted than LEDs that emit other wavelengths of light and a decrease in detection sensitivity be compensated for by illuminating light. This makes it possible to uniform detection sensitivity to each wavelength signal that is outputted from the imaging element.

TABLE 1

| | Emission output P₀ | Intensity of light |
|---|---|---|
| | (mW) · 50 mA | (μW/cm²) · 50 mA |
| NIR1 (780 nm) | 44.44 | 126 |
| NIR2 (850 nm) | 44.11 | 124 |
| NIR3 (940 nm) | 41.71 | 115 × 2 |

Furthermore, the light source 21 may emit, in addition to near-infrared light containing two or more wavelength components, a non-glaring visible light of, for example, 10 lux or lower. When used alone, such low-intensity visible light makes it difficult to take a fundus image or other images, as it causes image blur or noise increase. On the other hand, when used in combination with near-infrared light containing two or more wavelength components, such low-intensity visible light makes it possible to, while reducing glare during photographing, obtain more information about the condition of the fundus of the subject's eye 1 through the near-infrared light and the visible light.

The spectral element 24 serves to reflect a portion of the near-infrared light emitted from the light source 21 and emits the reflection toward the subject's eye 1, and usable examples of the spectral element 24 include a beam splitter and similar spectral elements. It should be noted that the condenser lens 23, which concentrates illuminating light (near-infrared light), a polarizing sheet (not illustrated) for removing a reflected image of the light source, and a mask (not illustrated) for forming a shape of illumination may be placed between the light pipe 22 and the spectral element 24. In that case, it is preferable to use, as the polarizing sheet, a wire grid polarizer that is compatible with near-infrared light.

The objective lens 25 serves to concentrate the near-infrared light, which is illuminating light, onto the subject's eye 1, and usable examples of the objective lens 25 include a biconvex lens and similar lenses. It should be noted that the objective lens 25 also has a role to concentrate reflected light from the subject's eye 1 in the after-mentioned light receiving optical system.

### [Light Receiving Optical System 3]

The light receiving optical system 3 serves to form an image by concentrating reflected light from the fundus or interior of the subject's eye 1, and is constituted by the objective lens 25, the spectral element 24, a focusing lens 31, or other constituent elements. The near-infrared light containing two or more wavelength components, with which the subject's eye 1 was irradiated, is reflected off the fundus or interior of the eye, passes through the objective lens 25 and the spectral element 24, and is formed into an image by the focusing lens 31.

It should be noted that the aforementioned polarizing sheet may be provided not in the irradiation optical system 2 but in the light receiving optical system 3. This makes it possible to reduce a reflection on a lens or the surface of the eyeball or a reflection of reflected light from the subject's eye 1. Moreover, as is the case with the aforementioned irradiation optical system 2, usable examples of the polarizing sheet in this case include a wire grid polarizer that is compatible with near-infrared light and similar polarizing sheets.

### [Imaging Unit 4]

The imaging unit 4 serves to take a fundus image or an intraocular image formed by the light receiving optical system 3 and output an image signal for each wavelength component, and includes one or more imaging elements. FIGS. 3 to 5 are diagrams each schematically showing an example configuration of the imaging unit 4. The imaging unit 4 needs only be configured to be able to detect near-infrared light separately for each wavelength component and, for example, as shown in FIG. 3, may be configured to include a plurality of imaging elements 42a to 42c and a spectral element (prism) 41 that splits reflected light from the subject's eye 1 into specific wavelengths and emits them separately toward the imaging elements 42a to 42c.

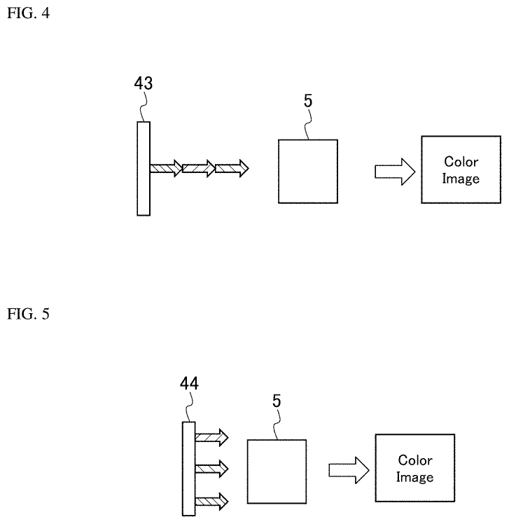

Alternatively, as shown in FIG. 4, the imaging unit 4 may be configured to irradiate the subject's eye with different wavelengths of near-infrared light either in sequence or in a time-sharing manner and take a fundus image or an intraocular image for each wavelength component by performing high-speed imaging at 30 FPS or higher through an imaging element 43. Alternatively, as shown in FIG. 5, the imaging unit 4 may be configured such that different wavelength components of light reflected by the subject's eye can be simultaneously detected through the use of an imaging element 44 including two or more types of pixels having different detection wavelengths. It should be noted that in any of the configurations of FIGS. 3 to 5, an optical signal detected by an imaging element is outputted to the image generation unit 5 as an image signal for each wavelength component.

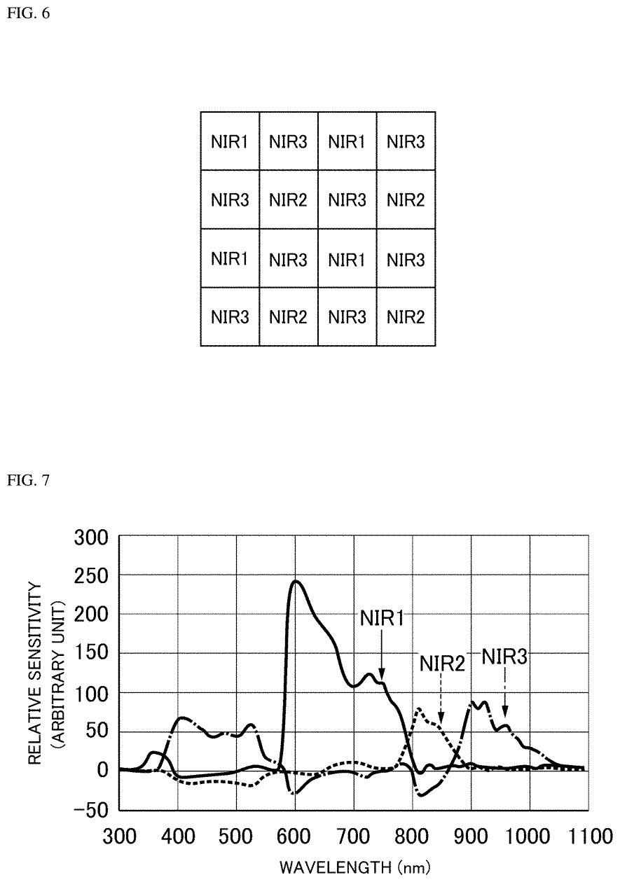

FIG. 6 is a diagram showing an example pixel arrangement of an imaging element having two or more types of pixels having different detection wavelengths, and FIG. 7 is a diagram showing the detection wavelength of each pixel shown in FIG. 6. The imaging element shown in FIG. 6 is provided with a first near-infrared pixel NIR1 that receives a first near-infrared light, a second near-infrared pixel NIR2 that receives a second near-infrared light differing in center wavelength from the first near-infrared light, and a third near-infrared pixel NIR3 that receives a third near-infrared light differing in center wavelength from the first near-infrared light and the second near-infrared light, and is capable of simultaneously detecting three types of near-infrared light having different center wavelengths. Using such an imaging element makes it possible to accurately detect a plurality of near-infrared lights with a simple apparatus configuration.

For example, the imaging element shown in FIG. 6 uses the first near-infrared pixel NIR1 to detect light in a near-infrared region that is correlative to red light (R), uses the second near-infrared pixel NIR2 to detect light in the near-infrared region that is correlative to blue light (B), and uses the third near-infrared pixel NIR3 to detect light in the near-infrared region that is correlative to green light (G). This enables the image generation unit 5 to generate a color image that is similar to a color photograph based on visible light.

Note here that as shown in FIG. 7, the light in the near-infrared region that is correlative to the red light (R), the light in the near-infrared region that is correlative to the blue light (B), and the light in the near-infrared region that is correlative to the green light (G) are light of any wavelength falling within a range of 700 to 830 nm, light of any wavelength falling within a range of 830 to 880 nm, and light of any wavelength falling within a range of 880 to 1200 nm, respectively, and are each light of a different wavelength.

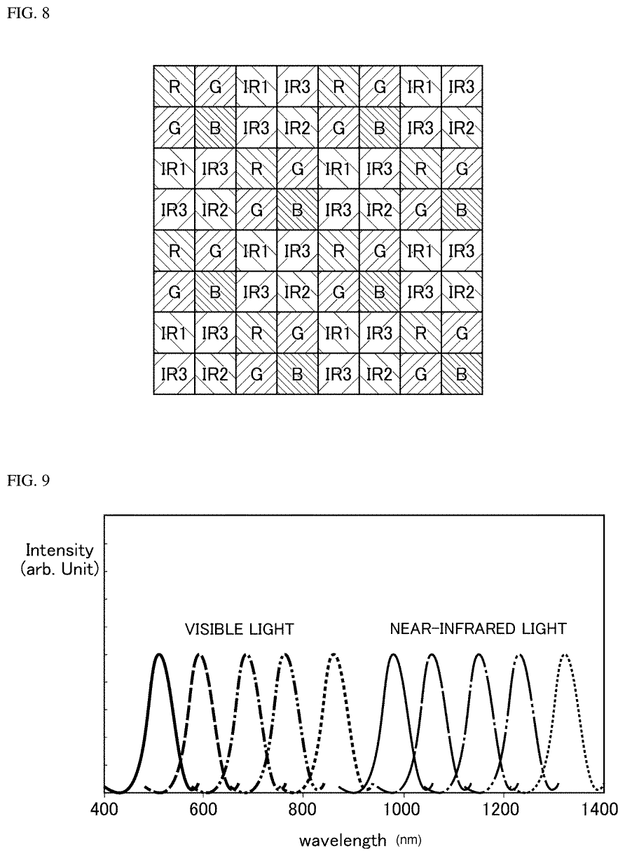

It should be noted that the imaging unit 4 is not limited to the aforementioned configurations, but it is possible to use a solid-state imaging element and a solid-state imaging device, described in PCT/JP2018/006193 or PCT/JP2018/017925, that are capable of simultaneously detecting a plurality of near-infrared lights having different wavelengths. FIG. 8 is a diagram showing an example pixel arrangement of an imaging element that is capable of detecting both visible light and near-infrared light, and FIG. 9 is a diagram showing the detection wavelength of the imaging element shown in FIG. 8. For example, in a case where the light source 21 used radiates low-intensity visible light together with near-infrared light, it is only necessary to use an imaging element that detects visible light together with near-infrared light through the aforementioned first, second, and third near-infrared pixels NIR1, NIR2, and NIR3 or an imaging element in which, as shown in FIG. 8, pixels that detect visible light (such as R, G, and B) are provided separately from near-infrared pixels.

The use of such an imaging element that is capable of detecting both visible light and near-infrared light makes it possible to, as shown in FIG. 9, simultaneously detect a plurality of lights ranging from a visible region to a near-infrared region. Alternatively, the imaging unit 4 may be provided with an imaging element that detects near-infrared light and an imaging element that detects visible light and configured to distribute reflected light from the subject's eye 1 to these imaging elements through the use of a spectral element.

### [Image Generation Unit 5]

The image generation unit 5 serves to generate a fundus image or an intraocular image of the subject's eye 1 by combining image signals outputted from the imaging unit 4. For example, in a case where the imaging unit 4 has detected light in a near-infrared region that is correlative to red light (R), blue light (B), and green light (G), the image generation unit 5 generates a color image by using an image signal from the first near-infrared pixel NIR1 as a red signal, using a signal from the second near-infrared pixel NIR2 as a blue signal, and using a signal from the third near-infrared pixel NIR3 as a green signal.

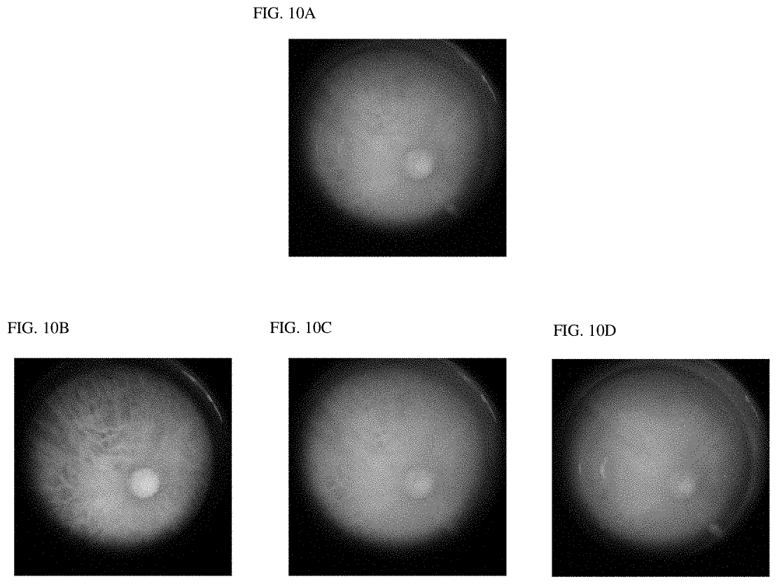

It should be noted that the image generation unit 5 may generate a fundus image or an intraocular image for each wavelength component in addition to a composite image. Observing a fundus image or an intraocular image for each wavelength component together with a color image makes it easier to detect an abnormality or a lesion in the fundus and other ophthalmic information. FIGS. 10A to 10D are fundus images taken by the ophthalmic photography apparatus of the first embodiment of the present invention. FIG. 10A is a color composite image, and FIGS. 10B to 10D are images based on near-infrared lights NIR1, NIR2, and NIR3 shown in FIG. 7, respectively.

As shown in FIG. 10A, using the ophthalmic photography apparatus of the present embodiment makes it possible to obtain, by near-infrared light photographing, a color fundus image that is similar to a photography based on irradiation with visible light. Further, since the color fundus image shown in FIG. 10A and the fundus images for each wavelength shown in FIGS. 10B to 10D are images taken at the same time, it is easy to compare abnormalities or lesions and identify their locations.

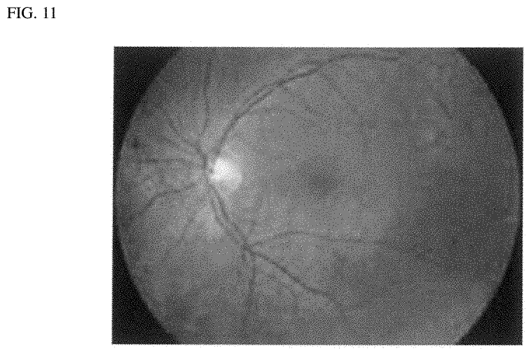

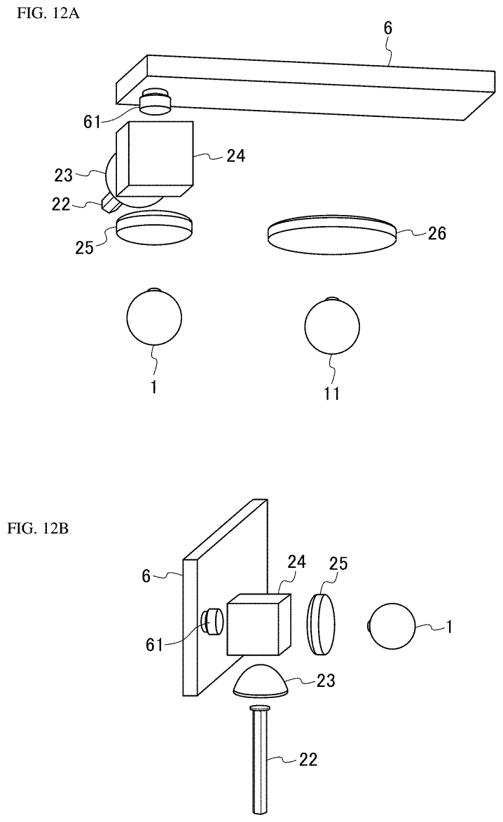

Further, the ophthalmic photography apparatus of the present embodiment is capable of taking a moving image as well as a still image. A visible light observation makes it difficult, because of glaring illuminating light, to take a moving image; however, in a case where near-infrared light is used as in the case of the present embodiment, it is possible to make a prolonged observation with a lighter burden on the subject. This makes it possible to take a moving image of the fundus or interior of the eye. FIG. 11 is a vascular observation image taken by the ophthalmic photography apparatus of the present embodiment. Taking a moving image of arteriocapillary tubes such as that shown in FIG. 11 makes it possible to observe a blood flow condition, thus making it possible to easily obtain ophthalmic information on the subject and information related to a physical condition such as a blood-pressure condition.

As described in detail above, the ophthalmic photography apparatus of the present embodiment, which takes a photograph with near-infrared light containing two or more wavelength components, makes it possible to reduce the burden on the subject as compared with visible light photographing. Since near-infrared light photographing can avoid pupil contraction, the ophthalmic photography apparatus of the present embodiment can be expected to reduce the number of retakes as compared with a conventional apparatus.

Further, by combining fundus images or intraocular images taken with two or more near-infrared lights having different wavelength components, the ophthalmic photography apparatus of the present embodiment can generate a color image that is similar to an image taken with visible light. As a result, through the use of the ophthalmic photography apparatus of the present embodiment, a fundus image or an intraocular image that makes it possible to easily confirm the presence or absence of an abnormality or a lesion can be obtained with only near-infrared light, which is less burdensome for the subject.

### (First Modification of First Embodiment)

Next, an ophthalmic photography apparatus according to a first modification of the first embodiment of the present invention is described. The constituent elements shown in FIG. 1, which may be provided in one apparatus, may be separately provided in two or more apparatuses and, for example, may be constituted by an optical member (photography kit) including the irradiation optical system 2 and the light receiving optical system 3 and an imaging device (camera) including the imaging unit 4 and the image generation unit 5.

FIGS. 12A and 12B are perspective views schematically showing a configuration of an ophthalmic photography apparatus of the present modification. In FIGS. 12A and 12B, constituent elements which are the same as those of the ophthalmic photography apparatus shown in FIG. 1 are given the same reference signs, and a detailed description of the constituent elements is omitted. As shown in FIGS. 12A and 12B, the ophthalmic photography apparatus of the present modification is provided with the image unit 4 and the image generation unit 5 in a smart device 6 including a camera function, and this smart device 6 has a camera 61 fitted with an optical member (photography kit) including a light source (not illustrated), the light pipe 22, the condenser lens 23, the spectral element 24, the objective lens 25, and a monitor screen observation lens 26.

In a case where a fundus image is taken through the use of the ophthalmic photography apparatus of the present modification, the smart device 6 and the optical member are placed, for example, so that the objective lens 25 is located in front of the left eye, which is the subject's eye 1, and the monitor screen observation lens 26 is located in front of an eye (right eye) 11 looking at a monitor. Moreover, the fundus of the left eye (subject's eye) 1 is photographed while an image is being checked by looking at the monitor of the smart device 6 with the right eye (eye looking at monitor) 11.

In so doing, by changing the display location of a fundus image on the monitor screen or displaying a mark on the monitor display to induce visual fixation, a visual point of the subject's eye 1 may be guided so that a fundus photography site is moved or the position of the eye can be adjusted so that the center of the fundus is aligned with the center of the monitor.

Thus, the ophthalmic photography apparatus of the present modification makes it possible to personally take a fundus image or an intraocular image, making it possible to quickly and easily observe the condition of an eye. It should be noted that constituent elements and effects of the present modification other than those described above are the same as those of the first embodiment described above.

### (Second Modification of First Embodiment)

Next, an ophthalmic photography apparatus according to a second modification of the first embodiment of the present invention is described. The ophthalmic photography apparatus of the present modification includes, in addition to the constituent elements of the ophthalmic photography apparatus of the first embodiment shown in FIG. 1, a data storage unit that stores a fundus image and an intraocular image and an image data processing unit that compares an image generated by the image generation unit 5 with an image stored in the data storage unit.

The ophthalmic photography apparatus of the present modification, which can compare an image taken in the past and stored in the data storage unit with an image taken most recently, allows the subject him/herself to easily grasp a change in the fundus or interior of the eye. It should be noted that constituent elements and effects of the present modification other than those described above are the same as those of the first embodiment described above and the first modification thereof.

### Second Embodiment

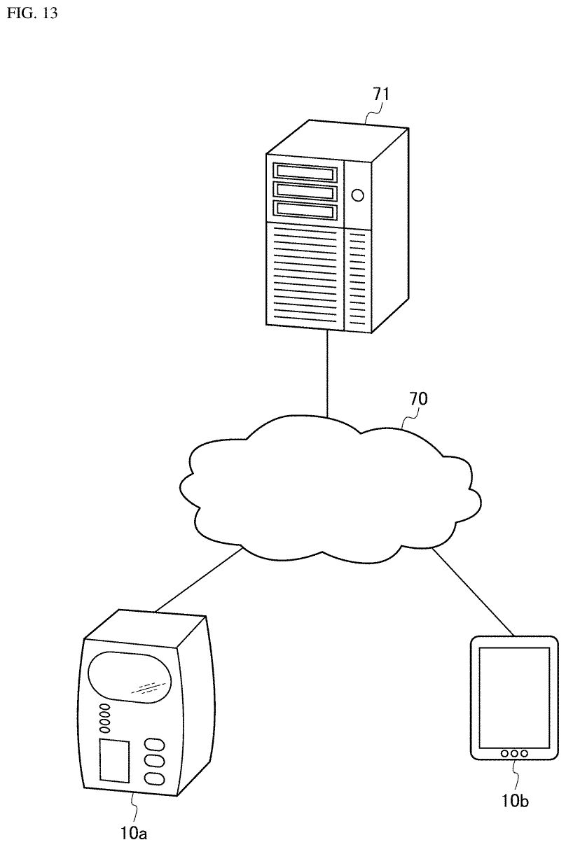

Next, an ophthalmic photography system of a second embodiment of the present invention is described. FIG. 13 is a diagram showing an overview of the ophthalmic photography system of the present embodiment. In the ophthalmic photography system of the present embodiment, as shown in FIG. 13, an ophthalmic photography apparatus (ophthalmic photography apparatus 10a) of the first embodiment shown in FIG. 1, an ophthalmic photography apparatus (ophthalmic photography apparatus 10b) of the first modification of the first embodiment shown in FIGS. 12A and 12B, and a server 71 are connected to one another via the Internet 70. In this server 71, fundus images and intraocular images taken in the past by the subject him/herself or a person other than the subject are stored as database information.

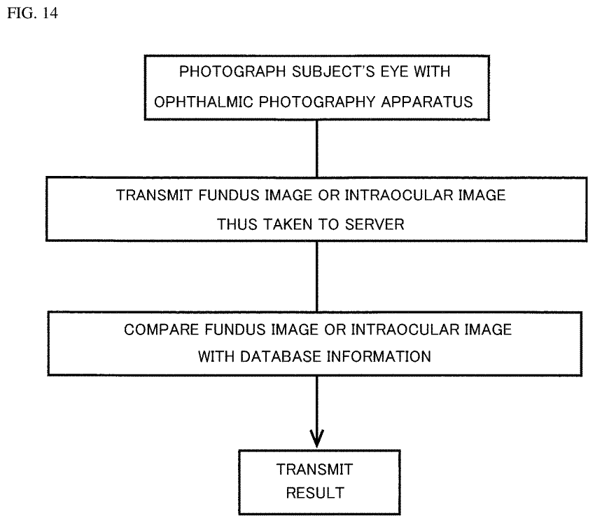

Next, operation of the ophthalmic photography system of the present embodiment is described. FIG. 14 is a flow chart showing how the ophthalmic photography system shown in FIG. 13 operates. As shown in FIG. 14, in a case where the ophthalmic photography system of the present embodiment is used to collect and process ophthalmic information, the subject's eye 1 is photographed by the ophthalmic photography apparatuses 10a and 10b first.

Then, the fundus image or the intraocular image thus taken is sent to the server 71. The server 71 compares the fundus image or the intraocular image thus taken with the database information, and transmits the result to the user (subject). It should be noted that a comparison result yielded by the ophthalmic photography system of the present embodiment may be checked by a doctor as needed and utilized in diagnosis.

The ophthalmic photography system of the present embodiment allows the subject him/herself to track changes in the fundus or interior of the eye on an as-needed basis by consecutively photographing the fundus or interior of the eye every day and transmitting the result to the server. This system can not only make a state observation but also detect a direction of change. Further, storing a pathological condition image in advance in the server makes it possible to compare fundus images (including moving images) through the Internet and check whether the subject's eye is in a pathological condition or a healthy condition. Even in a case where the subject's eye is in an intermediate condition between health and disease, this system makes it possible to perform a more accurate determination by using AI to compare accumulated image data.

Furthermore, the ophthalmic photography system of the present embodiment, which makes it possible to observe the fundus or interior of the eye with near-infrared light alone, hardly suffers from lens aberrations in measurements and makes it possible to take still images and moving images of the eye over a wide range including the fundus, the pupil, and the lens. In addition, this ophthalmic photography system makes it possible to comprehensively grasp information on the whole eye as well as the fundus, thus making it possible to discover an abnormal state of the eye that cannot be completely grasped by a fundus observation alone.

1. An ophthalmic photography apparatus comprising:
a light receiving optical system that forms an image by concentrating reflected light derived from near-infrared light reflected off a given location in a fundus or interior of the subject's eye;
an imaging unit that outputs an image signal for each wavelength component by taking a fundus image or an intraocular image formed by the light receiving optical system; and
an image generation unit that generates a fundus image or an intraocular image of the subject's eye by combining image signals outputted from the imaging unit,
wherein the imaging unit includes an imaging element that has two or more types of pixels having different detection wavelengths and that simultaneously detects two or more near-infrared lights having different center wavelengths.

2. (canceled)

3. The ophthalmic photography apparatus according to claim 1, wherein the imaging element has a first near-infrared pixel that receives a first near-infrared light, a second near-infrared pixel that receives a second near-infrared light differing in center wavelength from the first near-infrared light, and a third near-infrared pixel that receives a third near-infrared light differing in center wavelength from the first near-infrared light and the second near-infrared light.

4. The ophthalmic photography apparatus according to claim 3, wherein
the imaging element further has a first visible pixel that receives a first visible light, a second visible pixel that receives a second visible light differing in center wavelength from the first visible light, and a third visible pixel that receives third visible light differing in center wavelength from the first visible light and the second visible light,
the irradiation optical system radiates visible light either together with near-infrared light or separately from near-infrared light, and
the light receiving optical system forms an image of reflected light derived from the visible light.

5. The ophthalmic photography apparatus according to claim 1, wherein the pixels are provided on an identical element.

6. The ophthalmic photography apparatus according to claim 1, wherein the pixels are provided on different elements for each detection wavelength,
the ophthalmic photography apparatus further comprising a spectral element that splits light reflected off the fundus or interior of the subject's eye and emits the light thus split separately toward the pixels.

7. The ophthalmic photography apparatus according to claim 1, further comprising an irradiation optical system that irradiates a subject's eye with near-infrared light containing two or more wavelength components,
wherein the irradiation optical system includes a light source that simultaneously emits two or more near-infrared lights having different center wavelengths.

8. The ophthalmic photography apparatus according to claim 7, wherein
the irradiation optical system includes a light pipe that homogenizes and emits incoming light, and
the subject's eye is irradiated with light from the light source via the light pipe.

9. The ophthalmic photography apparatus according to claim 1, further comprising:
a data storage unit in which a fundus image and/or an intraocular image is/are stored; and
an image data processing unit that compares an image generated by the image generation unit with an image stored in the data storage unit.

10. An ophthalmic photography system comprising:
the ophthalmic photography apparatus according to claim 1; and
a server in which a fundus image and/or an intraocular image is/are stored,
the ophthalmic photography system comparing an image taken by the ophthalmic photography apparatus with an image stored in the server.

11. An ophthalmic photography apparatus, comprising:
a light receiving optical system that forms an image by concentrating near-infrared light emitted from a given location in a fundus or interior of a subject's eye;
an imaging unit that outputs an image signal for each wavelength h component by taking a fundus image or an intraocular image formed by the light receiving optical system; and
an image generation unit that generates a fundus image or an intraocular image of the subject's eye by combining image signals outputted from the imaging unit.
